(19) United States
(12) Patent Application Publication
(10) Pub. No.: US 2003/0073646 A1
(43) Pub. Date: Apr. 17, 2003

(54) Pharmaceutical compositions primarily for the treatment of genitourinary infections

(76) Inventor: Marton Milankovits, Budapest (HU)

Correspondence Address: HAUGEN LAW FIRM PLLP, 1130 TCF Tower, 121 South Eighth Street, Minneapolis, MN, 55402, US

(21) Appl. No.: 10/192,345

(22) Filed: Jul. 10, 2002

Related U.S. Application Data
(63) Continuation-in-part of application No. 08/776,273, filed on Jan. 22, 1997, now Pat. No. 6,432,935.

(51) Int. Cl. A61K 33/22; A61K 31/7048; A61K 31/704; A61K 31/496; A61K 31/4709

(52) U.S. Cl. 514/28; 514/29; 514/152; 514/192; 424/658; 514/298; 514/35; 514/253.08; 514/254.07

(57) ABSTRACT

Compositions having synergistic effective amounts of one or more antibacterial agents, a nitroimidazole, and an antifungal agent effective against a Candida species. The compositions are particularly useful in the treatment of genitourinary infections.

### CROSS-REFERENCE TO RELATED APPLICATIONS

This application is a continuation-in-part (CIP) of co-pending U.S. patent application Ser. No. 08/776,273, filed Jan. 22, 1997, the contents of which are herein incorporated by reference.

### FIELD OF THE INVENTION

This invention relates to novel compositions and their uses in treatments as suppositories, especially vaginal suppositories, ointments, vaginal drops and talc powders, and painting solutions, or any form of the compositions useful for systemic treatment.

### BACKGROUND OF THE INVENTION

A variety of vaginal suppositories are currently commercially available for the treatment of various maladies. The attending physician ordinarily decides which composition is best suited to the patient's needs following physical examination.

For example, for the treatment of vaginal mycosis, Canesten (active ingredient is clotrimazol; bis-phenyl-(2-chlorophenyl)-1-(imidazolyl)-methane; and Pimafucin (the active ingredient is natamycin-primaricin) are most commonly used. For fungal and protozoan infection, Klion D (the active ingredient is metronidazole; 1-(2′-hydroxiethyl)-2-methyl-5-nitroimidazol and myconasol-nitrate) is used. For protozoa infection, Klion vaginal suppository (active ingredient is metronidazole) is commonly used.

Certain compositions exert their effects through the disinfective action of iodine. These include Betadine (iodine is released from the carrier). Other vaginal suppositories feed the natural flora of the vagina. These include Genia 92 nutrients, e.g.: folic acid, lactic acid, lactose, and lactamine.

A common disadvantage of the above compositions is that none of them makes possible the combination of the effects of (i) bactericide (for: aerobe and anaerobe bacteria), involving anti-Mobiluncus and anti-Gardnerella, (ii) fungicide, and (iii) anti-protozoa simultaneously. Moreover, they have no antiviral effect at all.

### SUMMARY OF THE INVENTION

The invention relates to compositions which make possible the attack of pathogens from different directions, which simultaneously aid in the body's antiviral struggle. The invention facilitates rapid and simple selection of the safest and most useful compositions.

The basis of the invention is the discovery that a unique combination of active ingredients has numerous advantages over the art. In a preferred embodiment, the composition of the present invention includes an antibacterial agent, an antifungal agent effective against a candida species, and a nitroimidazole, wherein the antibacterial agent, the antifungal agent, and the nitroimidazole are present in the composition in synergistic effective amounts. Preferably, the composition of the present invention further includes a pharmaceutically acceptable carrier.

The basis of the discovery according to the invention is that the effect of the antibacterial agent ingredient is unexpectedly intensified by the other active components of the present composition. For example, the antibacterial effect, particularly of chloramphenicol and sulfonamide, against Chlamydia is greatly increased by the present compositions. Additionally, an increased inhibitory effect of the antibacterial agent, particularly chloramphenicol, and nitroimidazole, particularly metronidazole, components against anaerobe pathogens (e.g. B. fragilis) was observed when utilized in compositions of the present invention. The antibacterial effect of the sulfonamide and nitroimidazole also unexpectedly potentiated antibiotics generally against each pathogenic bacterium, in the antifungal protection provided by the antifungal agent.

The unexpected increased effectiveness of the components of the present composition when present in synergistic effective amounts has been observed when utilizing the three ingredients listed above. A further aspect and embodiment of the present invention provides for the inclusion of a sulfonamide in addition to another antibacterial agent, the antifungal agent, and the nitroimidazole, when present in a composition in synergistic effective amounts. As such, another embodiment of the present invention comprises an antibacterial agent, a sulfonamide, an antifungal agent effective against a candida species, and a nitroimidazole, wherein each of these components are present in a composition in synergistic effective amounts. Preferably, the composition further includes a pharmaceutically acceptable carrier for obtaining a suitably deliverable medication to a patient.

It has been observed through experimental data that the antibacterial effect of sulfonamide standing alone is greatly enhanced when coupled with the other ingredients of the present compositions. Therefore, such an unexpected increased effectiveness of sulfonamide when incorporated into the compositions of the present invention forms an additional embodiment and aspect of the present invention.

An additional aspect and embodiment of the present invention further supports the unexpected increased effectiveness of sulfonamide when combined with other active ingredients. A particular example which has proven unexpectedly effective against bacterial vaginosis is the combination of a sulfonamide and a nitroimidazole.

Thus, the treatment spectrum is broader and the effect of the combination is much stronger than would be expected from its individual components, while simultaneously decreasing the necessary dosages for treatment compared to the individual active ingredients. This results in a decrease of the possible side effects while using the present combination of elements. Another significant advantage of the solution according to the invention is that drug resistance does not occur.

### DETAILED DESCRIPTION OF THE INVENTION

The present invention relates to novel compositions having synergistic effective amounts of one or more antibacterial agents, a nitroimidazole, and an antifungal agent effective against a candida species. The antibacterial agents used in the present invention are also commonly referred to as antibiotics. The present invention contemplates the use of any antibiotic as defined by Martindale—The Extra Pharmacopoeia, 29^{th} Ed, London, The Pharmaceutical Press, 1989 (hereinafter “MARTINDALE”). As stated in MARTINDALE, page 94:

Antibiotics have traditionally been divided into bacterial static antibiotics which reversibly inhibit the growth of susceptible microorganisms and bactericidal antibiotics which kill the organisms in vitro. Given in high therapeutic doses, the aminoglycosides, cephalosporins, penicillins, and polymyxins are generally bactericidal by this criterium, whereas chloramphenicol, erythromycin, the sulfonamides, and the tetracyclines are usually bacteriostatic. However, an antibiotic which is bactericidal in a certain concentration may become bacteriostatic at lower concentrations.

In addition, antibiotics are classified into five classes based on chemical structure and mechanism of action in chapter 43 of Goodman & Gilman's The Pharmacological Basis of Therapeutics, 9^{th} Ed. (McGraw-Hill, 1996) (hereinafter “GOODMAN-GILMAN”). Such classes are set forth in Chapter 43, pages 1029-1030 of GOODMAN-GILMAN. As described therein, the individual members of each class are interchangeable with one another since they act on the target microorganism in the same way, and in most cases are also chemically interrelated with one another.

Experimental data set forth in the examples detailed below indicate that antibiotics belonging to each of the five classes defined in Chapters 43-48 of GOODMAN-GILMAN are effective and preferred for use in compositions of the present invention.

In some embodiments of the present invention, it is desired to utilize a sulfonamide for the particularly desirable antibiotic effect thereof when used in combination with the other components of the composition of the present invention. Any sulfonamide may be utilized in the composition of the present invention as all sulfonamides are structural analogs and competitive antagonists of para-aminobenzoic acid (see GOODMAN-GILMAN page 1058) Though all sulfonamides are interchangeable as applied to the composition of the present composition, a particularly preferred sulfonamide is sulfadimidin. In applications where a patient has an allergy to sulfonamides, it is preferred to formulate the composition of the present invention without inclusion of a sulfonamide.

The antifungal agents utilized in the compositions of the present invention are preferably effective against candida species (candida albicans, candida stelloidea, etc). Antifungal agents are divided into groups in GOODMAN-GILMAN, chapter 49, pages 1175-1190, defining antifungal agents effective against candida species which act only topically, and those agents effective against candida species both systemically and topically. The antifungal agents effective against candida species which act both systemically and topically are divided into the following subclasses: polyenes, azoles, and pyrimidines. According to GOODMAN-GILMAN, chapter 49, members of each subclass are interchangeable, in that each member of a respective subclass exhibits similar chemical properties and functionalities. Characteristic representatives of the polyenes are natamycin and nystatin. A characteristic representative of the azole subclass is clotrimazole. A characteristic representative of the pyrimidine subclass is flucitozine.

Another subclass of antifungal agents effective against candida species are those antifungal agents which act only topically. Characteristic representatives of this subclass are ciclopirox olamine, naftifine, terbinafine, and haloprogin. Though the antifungal agents selected for use in the compositions of the present invention are preferably effective against candida species, it is not necessary that such antifungal agents be effective solely against such candida species. For example, particular selected antifungal agents of the present invention may be effective against a multiplicity of fungi.

The nitroimidazole component of the compositions of the present invention further contribute to the unexpected increased efficacy of each component of the compositions. The mechanism of action of the nitroimidazoles is set forth in GOODMAN-GILMAN, page 996 as reflecting a selective toxicity to anaerobic or microaerophilic microorganisms. As such, the members of the nitroimidazole group share a common functionality and are so closely related in structure so as to be interchangeable (see GOODMAN-GILMAN). Characteristic representatives of the nitroimidazole group are metronidazole and tinidazole.

In a preferred embodiment, the composition of the present invention includes:

| Component | | Amount |
|---|---|---|
| a) | Antibacterial Agent | 0.04-0.30 g; |
| b) | Antifungal Agent | 0.025-0.30 g; |
| c) | Nitroimidazole | 0.10-0.45 g. |

A preferred pharmaceutically-acceptable carrier for the compositions of the present invention is polyethylene-glycol, but other suitable carriers may also be employed. The amount of polyethylene-glycol supplements the combination of the active ingredients to the necessary amount in case of a 10 unit package.

The essence of the present invention is the unexpected increased efficacy of the presently disclosed components when utilized in the compositions of the present invention, as contrasted with the minimal effectiveness of such components standing alone. Commercially available medications containing only one component of the compositions of the present invention have been found to be ineffective, even after extended treatment periods. Monotherapy, which is the treatment with a single active ingredient, is ineffective, in that a relapse rate of vaginal bacterial infections is about 80% of the patients receiving such monotherapy, even over a treatment duration of up to 20 days.

Through clinical trials, Applicant has discovered an unexpected synergy of effectiveness in compositions consisting of the three active ingredients referred to above.

The combination of elements used in the compositions of the present invention are more effective and qualitatively different from the separate administration of the individual components. Such compositions are preferably contained and administered in the form of vaginal suppositories, ointments, vaginal drops, talc powders, and painting solutions. Administration of the present compositions has resulted in complete recovery in cases when recovery could not be reached by the separate administration of the components. The compositions are useful in a variety of applications and treatments, including the following:

Prophylactic Use

Use of the present compositions prevent infection from infected swimming-pool water or sexual activity. The compositions are indispensable prior to gynecological operations (especially utero-vaginal interventions) as a prophylactic suppository.

Treatment of Infection

For infections, use of a preparation containing a composition of the present invention results in absolute recovery in 90% of the cases. Since systemic treatment is not needed, a smaller dose is administered. Cessation of treatment results in side effects disappearing (for local treatments there were no side effects observed). Resistance of the pathogens against the components used is also obviated because in local treatment the relatively small amount of preparation applied absolutely kills the pathogens.

Treatment of Chronic Vaginitis

Presently, techniques such as laser surgery, conventional surgery cryocoagulation or electro-cauterisation are used for the treatment of the cervicalization, (ectopium) or the slightly positive epithelial differences (such as the slightly acetic acid positive epithelium, or the decrease of the iodine positivity), or slightly pathological epithelium and P₃ cytological findings respectively.

The compositions of the present invention promote the spontaneous healing of the bleeding, inflamed portion of the uterus. As a result, the above-described invasive conventional treatments become unnecessary. Constant inflammation plays a decisive role in the formation of cancer of the cervix of the uterus. Application of the compositions according to the invention greatly diminishes the risk of the formation of the cancer of the cervix of the uterus, by stopping the inflammation. Nitroimidazole has a certain anticancer effects, which are observed during the radiation treatment of the tumors, where it increases the efficacy of the radiation treatment. In addition, it has been postulated that sulfonamides display anti-tumor characteristics as well (see Supuran C T et al. Carbonic Anhidrase Inhibitors: Sulfonamides as Antitumor Agents? Biorg. Med. Chem. 2001 March, 9(13):703-714).

Following the treatment with the present compositions, the laboratory findings improved from P₃ cytological to P₂ or P₁, without exception and the epithelium became normal kolposcopically.

Treatment of Complications of Ascending Infections

Ascending infections such as Cystitis, PID, etc., are typically caused by the same microbes as are in the vagina. The systemic application of the present combinations unexpectedly shorten the usual duration of treatment.

Antiviral Effect

The present combination acts against viral infections, possibly by killing all non-viral pathogens, enhancing the immunocapacity of the body against the viruses.

In those cases when, because of inflammation and vaginal discharge, the treatment of the Condyloma acuminatum (caused by the human papilloma virus) failed, local treatment using the present composition proved to be permanently successful.

Moreover, it was observed that patients with frequently reoccurring herpes genitalis, became permanently free of symptoms following the treatment with the preparation of the present invention.

The preparation according to the invention can also be administered to pregnant women to prevent adverse pregnancy outcome. In such applications, erythromycin is the preferred antibacterial agent component.

Application in the Veterinary Medicine

The treatment of kolpitis in female dogs by the present combinations proved similarly effective as compared to human treatment.

The compositions according to the invention were tested on 300-400 cases, with 300-400 controls. In all cases, the full microbiological examination included Chlamydia trachomatis and the Mycoplasmas.

Preparation of the compositions according to the invention can be carried out by methods known in the art for the preparation of such compositions.

The following experimental results demonstrate the unexpected efficacy of the compositions of the present invention, and set forth exemplary compositions indicative of the many possible species combinations useful in the treatment of various maladies. The examples will serve to further typify the nature of the invention, but should not be construed as a limitation on the scope thereof, which is defined solely by the appended claims.

### EXAMPLE 1

309 Patients
Seven days treatment

| | | |
|---|---|---|
| Chloramphenicol: | 0.10 | g |
| Sulfadimidin: | 0.10 | g |
| Nystatin: | 0.10 | g |
| Metronidazole: | 0.40 | g |
| Massa | 2.00 | g |
| Polyoxaethenum: | | |
| Completely treated: | 306 | |
| Remained the same: | 3 | |
| Worsened: | 0 | |

### EXAMPLE 2

90 Patients
Seven days treatment

| | | |
|---|---|---|
| Ciprofloxacin: | 0.04 | g |
| Sulfadimidin: | 0.10 | g |
| Nystatin: | 0.10 | g |
| Metronidazole: | 0.40 | g |
| Massa | 2.00 | g |
| Polyoxaethenum: | | |
| Completely treated: | 89 | |
| Remained the same: | 1 | |
| Worsened: | 0 | |

### EXAMPLE 3

77 Patients
Seven days treatment

| | | |
|---|---|---|
| Ampicillin: | 0.20 | g |
| Sulfadimidin: | 0.10 | g |
| Nystatin: | 0.10 | g |
| Metronidazole: | 0.40 | g |
| Massa | 2.00 | g |
| Polyoxaethenum: | | |
| Completely treated: | 75 | |
| Remained the same: | 2 | |
| Worsened: | 0 | |

### EXAMPLE 4

60 Patients
Seven days treatment

| | | |
|---|---|---|
| Sulfadimidin: | 0.10 | g |
| Nystatin: | 0.10 | g |
| Metronidazole: | 0.40 | g |
| Massa | 2.00 | g |
| Polyoxaethenum: | | |
| Completely treated: | 52 | |
| Remained the same: | 8 | |
| Worsened: | 0 | |

### EXAMPLE 5

19 Patients
Seven days treatment

| | | |
|---|---|---|
| Neomycin: | 0.10 | g |
| Sulfadimidin: | 0.10 | g |
| Nystatin: | 0.10 | g |
| Metronidazole: | 0.40 | g |
| Massa | 2.00 | g |
| Polyoxaethenum: | | |
| Completely treated: | 18 | |
| Remained the same: | 1 | |
| Worsened: | 0 | |

### EXAMPLE 6

10 Patients
Seven days treatment

| | | |
|---|---|---|
| Polymixin: | 0.05 | g |
| Sulfadimidin: | 0.10 | g |
| Nystatin: | 0.10 | g |
| Metronidazole: | 0.40 | g |
| Massa | 2.00 | g |
| Polyoxaethenum: | | |
| Completely treated: | 9 | |
| Remained the same: | 1 | |
| Worsened: | 0 | |

### EXAMPLE 7

12 Patients
Seven days treatment

| | | |
|---|---|---|
| Semicillin: | 0.20 | g |
| Sulfadimidin: | 0.10 | g |
| Nystatin: | 0.10 | g |
| Tinidazole: | 0.40 | g |
| Massa | 2.00 | g |
| Polyoxaethenum: | | |
| Completely treated: | 11 | |
| Remained the same: | 1 | |
| Worsened: | 0 | |

### EXAMPLE 8

10 Patients
Seven days treatment

| | | |
|---|---|---|
| Semicillin: | 0.20 | g |
| Sulfadimidin: | 0.10 | g |
| Clotrimazole: | 0.10 | g |
| Metronidazole: | 0.40 | g |
| Massa | 2.00 | g |
| Polyoxaethenum: | | |
| Completely treated: | 9 | |
| Remained the same: | 1 | |
| Worsened: | 0 | |

### EXAMPLE 9

10 Patients
Seven days treatment

| | | |
|---|---|---|
| Semicillin: | 0.20 | g |
| Sulfadimidin: | 0.10 | g |
| Natamycin: | 0.30 | g |
| Metronidazole: | 0.40 | g |
| Massa | 2.00 | g |
| Polyoxaethenum: | | |
| Completely treated: | 9 | |
| Remained the same: | 1 | |
| Worsened: | 0 | |

### EXAMPLE 10

10 Patients
Seven days treatment

| | | |
|---|---|---|
| Chloramphenicol: | 0.10 | g |
| Nystatin: | 0.10 | g |
| Metronidazole: | 0.40 | g |
| Massa | 2.00 | g |
| Polyoxaethenum: | | |
| Completely treated: | 8 | |
| Remained the same: | 2 | |
| Worsened: | 0 | |

### EXAMPLE 11

10 Patients
Seven days treatment

| | | |
|---|---|---|
| Semicillin: | 0.20 | g |
| Natamycin: | 0.30 | g |
| Metronidazole: | 0.40 | g |
| Massa | 2.00 | g |
| Polyoxaethenum: | | |
| Completely treated: | 7 | |
| Remained the same: | 3 | |
| Worsened: | 0 | |

### EXAMPLE 12

8 Patients
Seven days treatment

| | | |
|---|---|---|
| Chloramphenicol: | 0.10 | g |
| Sulfadimidin: | 0.10 | g |
| Nystatin: | 0.10 | g |
| Metronidazole: | 0.10 | g |
| Massa | 2.00 | g |
| Polyoxaethenum: | | |
| Completely treated: | 7 | |
| Remained the same: | 1 | |
| Worsened: | 0 | |

### EXAMPLE 13

Systemic administration (per os)
9 Patients
Seven days treatment

| | | |
|---|---|---|
| Ciprofloxacin: | 750 | mg/day (500 mg + 250 mg) |
| Ketoconazole: | 400 | mg/day (200 mg + 200 mg) |
| Tinidazole: | 1000 | mg/day (500 mg + 500 mg) |
| Completely treated: | 8 | |
| Remained the same: | 1 | |
| Worsened: | 0 | |

The above examples demonstrate the interchangeability of specific species within the respective genera antibacterial agent, nitroimidazole, and antifungal agent effective against a Candida species, while maintaining the efficacy of the various compositions of the present invention. Examples 10 and 11 illustrate the high level of effectiveness of even three component compositions of the present invention. Such effective experimental results are particularly unexpected when contrasted with the treatment efficacy using a single active ingredient. Such monotherapy treatments results in less than 20% complete recovery.

The compositions demonstrate significantly reduced treatment periods compared to application of these components separately. Previous treatment of the patients comprising the above test groups had failed with standard local administration of:

| The Agent | The Duration of Treatment | |
|---|---|---|
| Klion (metronidazole) | 10 | days |
| Klion D | 10 | days |
| Pimapucin (natamycin) | 20 | days |
| Canesten (clotrimazol) | 8-10 | days |

For example, it has been found that no benefit is realized through repeated 2000 mg doses of metronidazole administered alone (see Carey J C, Klebanoff M A, et al. Metronidazole to Prevent Pre-term Delivery in Pregnant Women with Asymptomatic Bacterial Vaginosis, New England Journal of Medicine 2000; 342: 534-40). In a further aspect of the present invention, it has been found that the respective effectiveness of a nitroimidazole and a sulfonamide when utilized alone is enhanced when combined. As such, an unexpected success rate of about 60% against bacterial vaginosis is achieved through the combination of a sulfonamide and a nitroimidazole. The following examples demonstrate the effectiveness of a pharmaceutical composition containing a sulfonamide and a nitroimidazole.

### EXAMPLE 14

10 Patients
Seven days treatment

| | | |
|---|---|---|
| Sulfadimidin: | 0.10 | g |
| Metronidazole: | 0.40 | g |
| Massa | 2.00 | g |
| Polyoxaethenum: | | |
| Completely treated: | 6 | |
| Remained the same: | 4 | |
| Worsened: | 0 | |

### EXAMPLE 15

12 Patients
Seven days treatment

| | | |
|---|---|---|
| Sulfadimidin: | 0.10 | g |
| Metronidazole: | 0.40 | g |
| Natrium | 0.05 | g |
| Tetraboratum | | |
| Massa | 2.00 | g |
| Polyoxaethenum: | | |
| Completely treated: | 8 | |
| Remained the same: | 4 | |
| Worsened: | 0 | |

Before treatment using the present combinations, vaginal bacterial samples were taken with cotton swabs (for culture and gram stain). The most common microorganisms found were E. coli, Enterococcus faecalis, B-group streptococci, Candida albicans, Ureaplasma, Mycoplasma hominis, Gardnerdella, Trichomonas vaginalis, Chlamydia trachomatis, and, to a lesser extent, Staphylococci, Proteus, Klebsiella, Haemophylus, etc. Antibiotic sensitivity was examined as well. After taking samples, patients were treated with the above-listed combinations of the present invention.

In addition to providing an unexpected increase in efficacy of each individual component when utilized in the combinations of the present invention, a significantly lower amount of each active ingredient is needed in the present invention as compared to typical monotherapy formulations.

The effective treatment of bacterial vaginosis is especially important because of its serious clinical implications and morbidity such as post-hysterectomy vaginal cuff cellulitis, plasmacell endometritis. In pregnant women, such clinical implications include amniotic fluid infection, clinical chorioamnionitis, postpartum endometritis, premature rupture of the membranes, pre-term delivery, and low birth weight.

The compositions of the present invention further displayed unexpected anti-viral activity. For example, in cases where the treatment of Condyloma acuminatum (caused by the human papilloma virus) failed, treatment using the compositions of the present invention along with local Podophyllin treatment proved to be permanently successful in 66% of the cases treated. Moreover, it has been observed that patients with frequently recurring herpes genitialis became permanently free of symptoms following treatment with the preparations of the present invention in 75% of the cases tested.

The following additional examples are contemplated by the present invention for use in treatment compositions:

| Combination I: | | |
|---|---|---|
| Chloramphenicol | 0.08 | g |
| Sulfadimidin | 0.2 | g |
| Clotrimazol | 0.15 | g |
| Metronidazole | 0.40 | g |
| Massa polyoxaethenum | 2.0 | g |
| Combination II: | | |
| Chloramphenicol | 0.1 | g |
| Sulfadimidin | 0.1 | g |
| Clotrimazol | 0.1 | g |
| Metronidazole | 0.40 | g |
| Massa polyoxaethenum | 2.0 | g |
| Combination III: | | |
| Unasyn | 0.1 | g |
| Sulphadimidin | 0.1 | g |
| Nystatin | 0.1 | g |
| Metronidazole | 0.4 | g |
| Massa polyoxaethenum | 2.0 | g |
| Combination IV: | | |
| Augmentin | 0.1 | g |
| Sulphadimidin | 0.1 | g |
| Nystatin | 0.1 | g |
| Natrium-tetraborat | 0.05 | g |
| Metronidazole | 0.4 | g |
| Massa polyoxaethenum | 2.0 | g |
| Combination V: | | |
| Oxacillin | 0.1 | g |
| Sulphadimidin | 0.1 | g |
| Nystatin | 0.1 | g |
| Metronidazole | 0.4 | g |
| Massa polyoxaethenum | 2.0 | g |
| Combination VI: | | |
| Cefaclor (Ceclor) | 0.05 | g |
| Sulphadimidin | 0.1 | g |
| Nystatin | 0.1 | g |
| Metronidazole | 0.4 | g |
| Massa polyoxaethenum | 2.0 | g |
| Combination VII: | | |
| Gentamicin | 0.05 | g |
| Sulphadimidin | 0.1 | g |
| Nystatin | 0.1 | g |
| Metronidazole | 0.4 | g |
| Massa polyoxaethenum | 2.0 | g |
| Combination VIII: | | |
| Clarithromycin (Klacid) | 0.1 | g |
| Sulphadimidin | 0.1 | g |
| Nystatin | 0.1 | g |
| Metronidazole | 0.4 | g |
| Massa polyoxaethenum | 2.0 | g |
| Combination IX: | | |
| Chloramphenicol | 0.1 | g |
| Sulphadimidin | 0.1 | g |
| Nystatin | 0.1 | g |
| Natrium-tetraborat | 0.05-0.10 | g |
| Metronidazole | 0.4 | g |
| Massa polyoxaethenum | 2.0 | g |
| Combination X: | | |
| Clindamycin | 0.05 | g |
| Natrium-tetraborat | 0.05 | g |
| Sulphadimidin | 0.1 | g |
| Nystatin | 0.1 | g |
| Metronidazole | 0.4 | g |
| Massa polyoxaethenum | 2.0 | g |

The present combination can be used in applications such as suppositories, ointments, talc powder, solution, painting solutions, vaginal drops, or impregnated tampons.

For systemic treatment situations, the preparations contain the combination of active ingredients in the same ratio as described above for localized treatment.

Ointment preparations contain the combination of the active ingredients in the same ratio as in the vaginal suppository, together with ointment base, with yellow Vaseline and other components known per se, if required. This preparation is especially effective in case of tissue damage (diabetes melitus, burning, etc). The talc powder preparation contains the active ingredient combination in solid form, with carriers such as talc, etc. The painting solutions and vaginal drops are prepared with an appropriate organic solvent. The vaginal drop solutions are useful in pediatric gynecology; but can also be used for adults in adequate doses.

The present compositions can also optionally contain borax (NA₂B₄O₇.₄H₂O).

The pharmaceutical compositions mentioned above can be prepared by known techniques used in the preparation of the pharmaceutical compositions.

What is claimed is:

1. A pharmaceutical composition, comprising:
(a) one or more antibacterial agents;
(b) an antifungal agent effective against a candida species; and
(c) a nitroimidazole,
wherein said one or more antibacterial agents, said antifungal agent, and said nitroimidazole are present in synergistic effective amounts.

2. A pharmaceutical composition as in claim 1, further comprising a pharmaceutically acceptable carrier.

3. A pharmaceutical composition as in claim 1 wherein said one or more antibacterial agents include a sulfonamide.

4. A pharmaceutical composition as in claim 1, further comprising borax.

5. A pharmaceutical composition as in claim 1 wherein said composition is selected from the group consisting of vaginal suppositories, ointments, solutions, painting solutions, vaginal drops, powders, and impregnated tampons.

6. A pharmaceutical composition as in claim 1 wherein said one or more antibacterial agents are selected from the group consisting of chloramphenicol, erythromycin, oxytetracyclin, ampicillin, cyprofloxacin, neomycin, polymixin, unasyn, augmentin, oxycillin, cefaclor, gentamicin, clarithromycin, clindamycin, and sulfadimin.

7. A pharmaceutical composition as in claim 1 wherein said antifungal agent is selected from the group consisting of clotrimazol, natamycin, and nystatin.

8. A pharmaceutical composition as in claim 1 being particularly adapted for use in systemic treatment of ascending infections.

9. A pharmaceutical composition as in claim 1 comprising:
(a) 0.04-0.30 grams of said one or more antibacterial agents;
(b) 0.025-0.30 grams of said antifungal agent; and
(c) 0.10-0.45 grams of said nitroimidazole.

10. A pharmaceutical composition consisting essentially of:
(a) an antibacterial agent;
(b) an antifungal agent effective against a candida species;
(c) a nitroimidazole; and
(d) a pharmaceutically acceptable carrier,
wherein said antibacterial agent, said antifungal agent, and said nitroimidazole are present in synergistic effective amounts.

11. A pharmaceutical composition as in claim 10 wherein said antibacterial agent is a sulfonamide.

12. A pharmaceutical composition as in claim 10 wherein said composition is selected from the group consisting of vaginal suppositories, ointments, solutions, painting solutions, vaginal drops, powders and impregnated tampons.

13. A pharmaceutical composition as in claim 10 wherein said one or more antibacterial agents are selected from the group consisting of chloramphenicol, erythromycin, oxytetracyclin, ampicillin, cyprofloxacin, neomycin, polymixin, unasyn, augmentin, oxycillin, cefaclor, gentamicin, clarithromycin, clindamycin, and sulfadimin.

14. A pharmaceutical composition as in claim 10 wherein said antifungal agent is selected from the group consisting of clotrimazol, natamycin, and nystatin.

15. A pharmaceutical composition as in claim 10 being particularly adapted for use in systemic treatment of ascending infections.

16. A pharmaceutical composition consisting essentially of:
(a) a first antibacterial agent;
(b) a sulfonamide;
(c) an antifungal agent effective against a candida species;
(d) a nitroimidazole; and
(e) a pharmaceutically acceptable carrier,
wherein said antibacterial agent, said sulfonamide, said antifungal agent, and said nitroimidazole are present in synergistic effective amounts.

17. A pharmaceutical composition consisting essentially of:
(a) a sulfonamide;
(b) a nitroimidazole; and
(c) a pharmaceutically acceptable carrier.

18. A pharmaceutical composition as in claim 17, including borax.
